(19) United States
(12) Patent Application Publication
(10) Pub. No.: US 2010/0285567 A1
(43) Pub. Date: Nov. 11, 2010

(54) TAT SIGNAL PEPTIDES FOR PRODUCING PROTEINS IN PROKARYOTES

(76) Inventors: Kai Bao, Palo Alto, CA (US); Fernando Valle, Burlingame, CA (US)

Correspondence Address: DANISCO US INC.;ATTENTION: LEGAL DEPARTMENT, 925 PAGE MILL ROAD, PALO ALTO, CA, 94304, US

(21) Appl. No.: 12/664,357

(22) PCT Filed: Jun. 2, 2008
(86) PCT No.: PCT/US08/06943

§ 371 Date: May 12, 2010

Related U.S. Application Data
(60) Provisional application No. 60/936,186, filed on Jun. 18, 2007.
(60) Provisional application No. 60/936,183, filed on Jun. 18, 2007.

(51) Int. Cl. C12N 9/14; C12N 1/21; C12N 15/63; C07H 21/04

(52) U.S. Cl. 435/195; 435/252.35; 435/320.1; 536/23.2

(57) ABSTRACT

This invention provides polynucleotides encoding TAT fusion proteins, and methods for producing proteins of interest in a host cell. In particular, the present invention relates to polynucleotides, vectors, polypeptides and methods for expressing organophosphate-degrading enzymes e.g. organophosphorus hydrolase (OPH) in host cell, such as a Streptomyces species host cell.

This application claims priority to U.S. Provisional Application 60/936,183, filed Jun. 18, 2007, and U.S. Provisional Application 60/936,186 filed Jun. 18, 2007.

### GOVERNMENT SUPPORT

Portions of this work were funded by Contract No. BAA ECBC-04 with the Edgewood Chemical Biological Center (ECBC) with the U.S. Army. Accordingly, the United States Government may have certain rights in this invention.

### FIELD OF THE INVENTION

This invention provides polynucleotides encoding TAT fusion proteins, and methods for producing proteins of interest in microorganisms. In particular, the present invention relates to polynucleotides, vectors, polypeptides and methods for expressing organophosphate-degrading enzymes e.g. organophosphorus hydrolase (OPH) in a host cell, such as a Streptomyces species host cell.

### BACKGROUND

In prokaryotes two pathways for protein translocation across the cytoplasmic membrane have been recognized. In most bacteria the general secretory (Sec) pathway is the best-characterized route for protein export. Proteins exported by the Sec pathway are translocated across the membrane in an unfolded state through a membrane-embedded translocon to which they are targeted by cleavable N-terminal signal peptides.

The second general export pathway is the twin-arginine translocation (Tat) pathway. Unlike the Sec system, the Tat system is involved in the transport of pre-folded protein substrates. Proteins are targeted to the Tat pathway by possession of N-terminal signal peptides that include a conserved twin-arginine motif in the N-region of Tat signal peptide.

Because of its ability to secrete pre-folded protein substrates, the Tat pathway represents a significant mechanism for producing secreted proteins, and it can be exploited for large-scale production of proteins used to detoxify organophosphate compounds that are used in a variety of agricultural pesticides and as chemical warfare agents such as sarin, soman and VX [O-ethyl-S-(2-diisopropylaminoethyl) methylphosphonothioate].

### SUMMARY OF THE INVENTION

The present teachings are based, at least in part, on the discovery that certain proteins can be produced using the Tat pathway in bacterial host cells. Accordingly, the present invention provides polynucleotides encoding TAT fusion proteins, and methods for producing proteins of interest in a host cell. In particular, the present invention relates to polynucleotides, vectors, polypeptides and methods for expressing organophosphate-degrading enzymes e.g. organophosphorus hydrolase (OPH) in a host cell, such as a Streptomyces species host cell.

In one embodiment, the invention provides an isolated polynucleotide comprising a first nucleic acid sequence encoding a TAT signal peptide operably linked to a second nucleic acid sequence encoding a phosphoric triester hydrolase classified as a EC 3.1.8 protein. In another embodiment, the isolated polynucleotide of the invention comprises a first nucleic acid sequence encoding a TAT signal peptide having an amino acid sequence chosen from SEQ ID NOS:1-5, operably linked to second nucleic acid sequence encoding a phosphoric triester hydrolase classified as a EC 3.1.8 protein. In another embodiment, the isolated polynucleotide of the invention comprises a first nucleic acid sequence encoding a TAT signal peptide operably linked to a second nucleic acid sequence encoding an organophosphorus hydrolase (OPH). In another embodiment, the organophosphate hydrolase is an OPH of SEQ ID NO:18 or a variant thereof. In another embodiment, the invention provides an isolated polynucleotide comprising a first nucleic acid sequence encoding a TAT signal peptide operably linked to a second nucleic acid sequence encoding a phosphoric triester hydrolase classified as a EC 3.1.8 protein, wherein the second nucleic acid of the isolated polynucleotide is codon optimized for expressing the phosphoric triester hydrolase in a Streptomyces sp. host cell. In yet another embodiment, the invention provides an isolated polynucleotide comprising a first nucleic acid sequence encoding a TAT signal peptide operably linked to a second nucleic acid sequence encoding a phosphoric triester hydrolase classified as a EC 3.1.8, wherein the isolated polynucleotide is further operably linked to the A4 promoter of SEQ ID NO:23.

In another embodiment, the invention provides an expression vector that comprises an isolated polynucleotide comprising a first nucleic acid sequence encoding a TAT signal peptide operably linked to a second nucleic acid sequence encoding a phosphoric triester hydrolase classified as a EC 3.1.8 protein. In some embodiments, the expression vector comprises a first nucleic acid sequence encoding a TAT signal peptide of any one of SEQ ID NOS:1-5 and a second nucleic acid sequence encoding a phosphoric triester hydrolase classified as a EC 3.1.8 protein. In some embodiments, the expression vector comprises an isolated polynucleotide comprising a first nucleic acid sequence encoding a TAT signal peptide operably linked to a second nucleic acid sequence encoding an organophosphorus hydrolase. In some embodiments, the organophosphorus hydrolase is an OPH of SEQ ID NO:18 or a variant thereof. In some embodiments, the isolated polynucleotide comprising a first nucleic acid sequence encoding a TAT signal peptide that is operably linked to a second nucleic acid sequence encoding a phosphoric triester hydrolase is further operably linked to the A4 promoter of SEQ ID NO:23. In other embodiments, the nucleic acid sequence encoding the phosphoric triester hydrolase is codon optimized for expression in a Streptomyces sp. host cell. In some embodiments, the Streptomyces sp. host cell is a Streptomyces lividans host cell.

In another embodiment, the invention provides an isolated host cell that comprises an expression vector comprising an isolated polynucleotide comprising a first nucleic acid sequence encoding a TAT signal peptide operably linked to a second nucleic acid sequence encoding a phosphoric triester hydrolase classified as a EC 3.1.8 protein. In some embodiments, the host cell comprises an expression vector that comprises a first nucleic acid sequence encoding a TAT signal peptide of any one of SEQ ID NOS:1-5 and a second nucleic acid sequence encoding a phosphoric triester hydrolase. In another embodiment, the host cell comprises an expression vector comprising an isolated polynucleotide comprising a first nucleic acid sequence encoding a TAT signal peptide operably linked to a second nucleic acid sequence encoding an organophosphorus hydrolase. In some embodiments, the organophosphate hydrolase is an OPH of SEQ ID NO:18 or a variant thereof. In another embodiment, the expression vector comprised in the host cell comprises an isolated polynucleotide comprising a first nucleic acid sequence encoding a TAT signal peptide that is operably linked to a second nucleic acid sequence encoding a phosphoric triester hydrolase is further operably linked to the A4 promoter of SEQ ID NO:23. In some embodiments, the nucleic acid sequence encoding the phosphoric triester hydrolase is codon optimized for expression in a Streptomyces sp. host cell. In some embodiments, the host cell of the invention is a Streptomyces sp. host cell e.g. a Streptomyces lividans host cell.

In another embodiment, the invention provides an isolated fusion polypeptide comprising a TAT2 or a TAT3 signal peptide linked to the organophosphorus hydrolase of SEQ ID NO:18. In another embodiment, the TAT signal peptide comprised in the isolated fusion polypeptide is chosen from SEQ ID NO:1 or 5.

In another embodiment, the invention provides for a method for producing an organophosphate degrading enzyme comprising: expressing an isolated polynucleotide comprising a first nucleic acid sequence encoding a TAT signal peptide operably linked to a second nucleic acid sequence encoding a phosphoric triester hydrolase classified as a EC 3.1.8 protein; and producing said organophosphate degrading enzyme. In one embodiment, the organophosphate degrading enzyme produced by the method of the invention an organophosphorus hydrolase. In some embodiments, the organophosphorus hydrolase is an OPH of SEQ ID NO:18 or a variant thereof. In another embodiment, the method of the invention further comprises recovering the enzyme produced by the host cell. In another embodiment, the host cell of the method of the invention is a Streptomyces sp. host cell.

### BRIEF DESCRIPTION OF THE DRAWINGS

The skilled artisan will understand that the drawings are for illustration purposes only. The drawings are not intended to limit the scope of the present teachings in any way.

FIG. 1 shows a pKB105 vector containing an the Apergillus niger A4 promoter, a truncated signal sequence of S. lividans cellulase (CeIA), a polynucleotide encoding a cellulase 11AG8 gene from an Actinomyces species, and a cellulase 11AG3 terminator sequence.

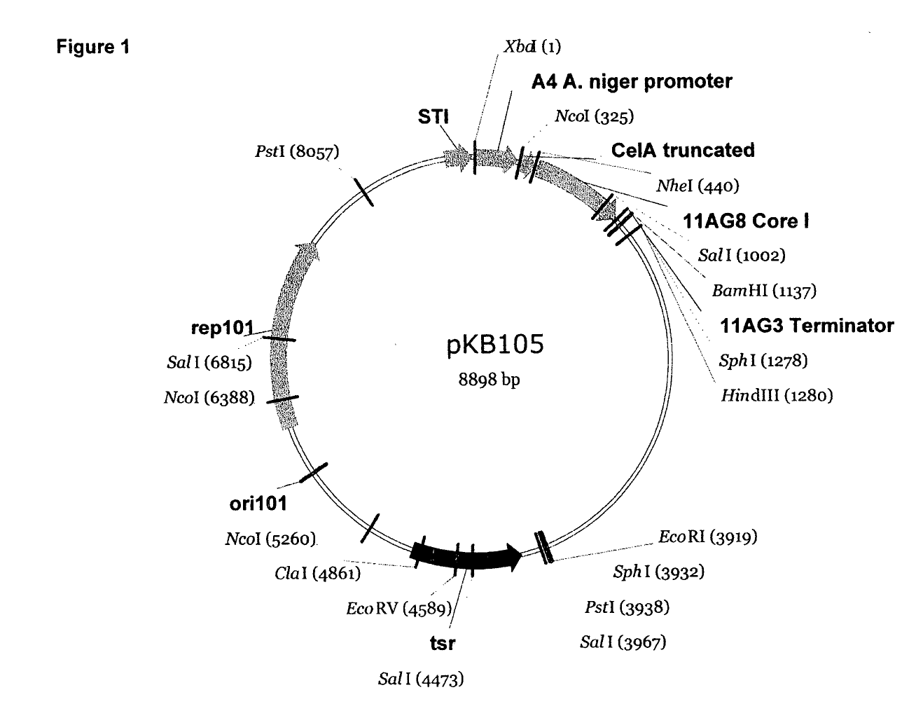

FIG. 2 shows a pKB229 vector derived form the pKB105 vector of FIG. 1 in which the CeIA signal peptide is replaced with a TAT1 signal sequence and the cellulase gene is replaced with the codon optimized OPH gene.

FIG. 3 shows a pKB231 vector derived form the pKB105 vector of FIG. 1 in which the CeIA signal peptide is replaced with a TAT2 signal sequence and the cellulase gene is replaced with the codon optimized OPH gene.

FIG. 4 shows a pKB233 vector derived form the pKB105 vector of FIG. 1 in which the CeIA signal peptide is replaced with a TAT3 signal sequence and the cellulase gene is replaced with the codon optimized OPH gene.

FIG. 5 shows a pKB234 vector that was derived from the pKB233 vector by removing the E. coli DNA sequences between the SphI and EcoRI restriction sites.

FIG. 6 shows SDS-PAGE analysis of OPH produced in Streptomyces and expressed in the host cells fused to TAT1, TAT2 and TAT3 signal peptides.

FIG. 7 shows the effect of addition of Zn²⁺ and Co²⁺ on the production and storage stability of OPH expressed in Streptomyces as a TAT3 fusion protein.

FIG. 8 shows the identification by tryptic in-gel digestion and MALDI peptide mass mapping of the expected OPH produced by the Streptomyces cells

FIG. 9 shows the paraoxonase activity of OPH produced by Streptomyces host cells.

### DETAILED DESCRIPTION OF THE INVENTION

This invention provides polynucleotides encoding TAT fusion proteins, and methods for producing proteins of interest in a host cell. In particular, the present invention relates to polynucleotides, vectors, polypeptides and methods for expressing organophosphorus hydrolase (OPH) in a host cell, such as a Streptomyces species host cell.

The present teachings will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Markham, The Harper Collins Dictionary of Biology, Harper Perennial, NY (1991) provide those of skill in the art with a general dictionaries of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular “a”, “an” and “the” includes the plural reference unless the context clearly indicates otherwise. Numeric ranges are inclusive of the numbers defining the range. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The headings provided herein are not limitations of the various aspects or embodiments which can be had by reference to the specification as a whole. Accordingly, the terms defined immediately below are more fully defined by reference to the specification as a whole.

### DEFINITIONS

The term “organophosphate degrading enzymes” herein refers to enzymes that catalyses the hydrolysis of phosphoester bonds in organophosphates.

The terms “phosphoric triester hydrolase” or “phosphotriester” herein refer to an enzyme classified as EC3.1.8, which acts on organophosphorus compounds (such as paraoxon) including esters of phosphonic and phosphinic acids. Phosphoric triester hydrolases include aryldialkylphosphatases (EC 3.1.8.1), also known as OPH, and diisopropyl-fluorophosphatase (EC 3.1.8.2), also known as DFPase.

The term “polypeptide” as used herein refers to a compound made up of a single chain of amino acid residues linked by peptide bonds. The term “protein” as used herein may be synonymous with the term “polypeptide” or may refer, in addition, to a complex of two or more polypeptides. The conventional three-letter or single letter codes for amino acid residues are used herein wherein alanine (A); arginine (R); asparagine (N); aspartic acid (D); cysteine (C); glutamic acid (E); glutamine (Q); glycine (G); histidine (H); isoleucine (I); leucine (L); lysine (K); methionine (M); phenylalanine (F); proline (P); serine (S); threonine (T); tryptophan (W); tyrosine (Y) and valine (V).

The term “fusion polypeptide” or “Tat fusion polypeptide” as used herein refers to a Tat signal peptide linked either directly, or via a linker, to a protein of interest.

A “signal peptide” as used herein refers to an amino-terminal extension on a protein to be secreted. Nearly all secreted proteins use an amino-terminal protein extension which plays a crucial role in the targeting to, and translocation of, precursor proteins across the membrane and which is generally proteolytically removed by a signal peptidase during or immediately following membrane transfer.

A “Tat signal peptide” refers to an N-terminally extended sequence which includes two consecutive arginine residues and which functions in the secretion of proteins in a prefolded confirmation. A “Tat signal peptide” may be interchangeably referred to as “Tat peptide” or “Tat polypeptide”.

As used herein, a “protein of interest” or “polypeptide of interest” refers to the protein to be expressed and secreted by the host cell. The protein of interest may be any protein which up until now has been considered for expression in prokaryotes. The protein of interest may be either homologous or heterologous to the host. In the case of a homologous protein of interest, over expression is expression above normal levels in said host. In the case of a heterologous protein of interest, any expression is over expression.

As used herein, the term “heterologous protein” refers to a protein or polypeptide that does not naturally occur in a host cell. Examples of heterologous proteins include, but are not limited to, enzymes, such as hydrolases, including esterases, proteases, glycosylases; isomerases, such as racemases, epimerases, tautomerases, or mutases; lyases; ligases; transferases, such as kinases, transaminases and phosphotransferases; or oxidoreductases, such as oxidases and dehydrogenases. The heterologous gene may encode therapeutically significant proteins or peptides, such as growth factors, cytokines, ligands, receptors and inhibitors, as well as vaccines and antibodies. The gene may encode commercially important industrial proteins or peptides, such as esterases, proteases, carbohydrases such as amylases and glucoamylases, cellulases, oxidases and lipases. The gene of interest may be a naturally occurring gene, a mutated gene or a synthetic gene.

The term “homologous protein” refers to a protein or polypeptide native or naturally occurring in a host cell. The invention includes homologous proteins that are variants, e.g., comprising an insertion, deletion or interruption, as compared to the naturally occurring homologous protein.

The term “nucleic acid” and “polynucleotide” are used interchangeably and encompass RNA, DNA and cDNA molecules. As used herein, the term refers to a polymeric form of nucleotides of any length, either ribonucleotides or deoxyribonucleotides. The term refers only to the primary structure of the molecule and thus includes double- and single-stranded DNA and RNA. It also includes known types of modifications, include, but are not limited to, labels which are known in the art, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example proteins (including for e.g., nucleases, toxins, antibodies, signal peptides, poly-L-lysine, etc.), those with intercalators (e.g., acridine, psoralen, etc.), those containing chelates (e.g., metals, radioactive metals, boron, oxidative metals, etc.), those containing alkylators, those with modified linkages (e.g., alpha anomeric nucleic acids, etc.), as well as unmodified forms of the polynucleotide. Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene. Because the genetic code is degenerate, more than one codon may be used to encode a particular amino acid, and the present invention encompasses all polynucleotides, which encode a particular amino acid sequence.

A polynucleotide or a polypeptide having a certain percent (e.g., 65%, 70%, 75%, 80%, 85%, 90%, 95%, or 99%) of sequence identity with another sequence means that, when aligned, that percentage of bases or amino acid residues are the same in comparing the two sequences. This alignment and the percent homology or identity can be determined using any suitable software program known in the art, for example, those described in Current Protocols in Molecular Biology (F. M. Ausubel et al. (eds) 1987, Supplement 30, section 7.7.18). In some embodiments, the programs include the GCG Pileup program, FASTA (Pearson et al. (1988) Proc. Natl, Acad. Sci. USA 85:2444 2448), and BLAST (BLAST Manual, Altschul et al., Natl Cent. Biotechnol. Inf., Natl Lib. Med. (NCIB NLM NIH), Bethesda, Md., and Altschul et al., (1997) NAR 25:3389 3402). Other exemplary alignment programs are ALIGN Plus (Scientific and Educational Software, PA), preferably using default parameters, and the TFASTA Data Searching Program available in the Sequence Software Package Version 6.0 (Genetics Computer Group, University of Wisconsin, Madison, Wis.). One skilled in the art will recognize that sequences encompassed by the invention include those that hybridize under stringent hybridization conditions with the polynucleotides of the invention.

A “heterologous” nucleic acid construct or sequence, also referred to herein as a “chimeric gene,” has a portion of the sequence which is not native to the cell in which it is expressed. Heterologous, with respect to a control sequence refers to a control sequence (e.g., a promoter or an enhancer) that does not function in nature to regulate the same gene the expression of which it is currently regulating. Generally, heterologous nucleic acid sequences are not endogenous to the cell or part of the genome in which they are present, and have been added to the cell, by infection, transfection, microinjection, electroporation, or the like. A “heterologous” nucleic acid construct may contain a control sequence/DNA coding sequence combination that is the same as, or different from a control sequence/DNA coding sequence combination found in the native cell. The control sequence, e.g., a transcriptional regulatory sequence is typically operably linked to a heterologous protein coding sequence, or, in a selectable marker chimeric gene, to a selectable marker gene encoding a protein conferring antibiotic resistance to transformed cells. A typical chimeric gene or heterologous nucleic acid construct of the present invention, includes a transcriptional regulatory region, a signal peptide coding sequence, a protein coding sequence, and a terminator sequence. The transcriptional regulatory region could be constitutive or inducible.

As used herein, the term “vector” refers to a polynucleotide sequence designed to introduce nucleic acids into one or more cell types. Vectors include cloning vectors, expression vectors, shuttle vectors, plasmids, cassettes and the like.

As used herein, the term “expression vector” refers to a vector that has the ability to incorporate and express heterologous DNA fragment in a foreign cell. Many prokaryotic and eukaryotic expression vectors are commercially available.

As used herein, the terms “nucleic acid construct” and “expression vector” are used interchangeably to refer to nucleic acid used to introduce sequences into a host cell or organism. The nucleic acid may be generated in vitro by PCR or any other suitable technique(s) known to those in the art. The nucleic acid construct or recombinant expression cassette can be incorporated into a plasmid, chromosome, mitochondrial nucleic acid, plastid nucleic acid, virus, or nucleic acid fragment. Typically, the recombinant expression cassette portion of an expression vector or nucleic acid construct includes, among other sequences, a nucleic acid sequence to be transcribed and a promoter. In some embodiments, expression vectors have the ability to incorporate and express heterologous nucleic acid fragments in a host cell. Many prokaryotic and eukaryotic expression vectors are commercially available. Selection of appropriate expression vectors is within the knowledge of those having skill in the art.

As used herein, the term “plasmid” refers to a circular double-stranded (ds) DNA construct used as a cloning vector, and which forms an extrachromosomal self-replicating genetic element in many bacteria and some eukaryotes.

As used herein, the term “promoter” refers to a nucleic acid sequence that functions to direct transcription of a gene. The promoter will generally be appropriate to the host cell in which the target gene is being expressed. The promoter together with other transcriptional and translational regulatory nucleic acid sequences (also termed “control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

A nucleic acid is “operably linked” when it is placed into a functional relationship with another nucleic acid sequence. For example, nucleic acid encoding a secretory leader is operably linked to nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the nucleic acid sequences being linked are contiguous, and, in the case of a secretory leader, contiguous and in reading phase. However, enhancers and promoters do not have to be contiguous. Linking is accomplished by ligation at convenient restriction sites. If such sites do not exist, synthetic oligonucleotide adaptors or linkers are used in accordance with conventional practice.

A first polypeptide “linked” to a second polypeptide generally means that the polypeptide sequences being linked are contiguous and form a fusion protein.

As used herein, the term “gene” refers to a polynucleotide (e.g., a DNA segment) involved in producing a polypeptide chain, that may or may not include regions preceding and following the coding region, e.g. 5′ untranslated (5′ UTR) or “leader” sequences and 3′ UTR or “trailer” sequences, as well as intervening sequences (introns) between individual coding segments (exons).

The term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified or that a protein is expressed in a non-native or genetically modified environment, e.g., in an expression vector for a prokaryotic or eukaryotic system. Thus, for example, recombinant cells express nucleic acids or polypeptides that are not found within the native (non-recombinant) form of the cell or express native genes that are otherwise abnormally expressed, under expressed, over expressed or not expressed at all.

As used herein, the terms “transformed”, “stably transformed” and “transgenic” used in reference to a cell means the cell has a non-native (e.g., heterologous) nucleic acid sequence integrated into its genome or as an episomal plasmid that is maintained through multiple generations.

As used herein, the term “expression” refers to the process by which a polypeptide is produced based on the nucleic acid sequence of a gene. The process includes both transcription and translation.

As used herein, the term “operably linked” means that the transcriptional and translational regulatory nucleic acid is positioned relative to the coding sequences in such a manner that transcription is initiated. Generally, this will mean that the promoter and transcriptional initiation or start sequences are positioned 5′ to the coding region. The transcriptional and translational regulatory nucleic acid will generally be appropriate to the host cell used to express the protein. Numerous types of appropriate expression vectors, and suitable regulatory sequences are known in the art for a variety of host cells.

The terms “production” and “secretion” with reference to a desired protein e.g. an OPH, encompasses the processes that follow expression and includes the processing steps of: removing the signal peptide, which is known to occur during protein secretion, and the translocation of the desired protein to the outside of the host cell. The term “processing or “processed” with reference to an OPH refers to the maturation process that a full-length protein e.g. an OPH, undergoes to become an active mature enzyme.

The term “under transcriptional control,” as used herein, indicates that transcription of a polynucleotide sequence, usually a DNA sequence, depends on its being operably linked to an element which contributes to the initiation of, or promotes transcription.

The term “under translational control,” as used herein, indicates a regulatory process which occurs after mRNA has been formed.

As used herein, the term “plasmid” refers to a circular double-stranded (ds) DNA construct used as a cloning vector, and which forms an extrachromosomal self-replicating genetic element in some eukaryotes or prokaryotes, or integrates into the host chromosome.

The term “introduced” in the context of inserting a nucleic acid sequence into a cell, means “transfection”, or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

The terms “recovered,” “isolated,” and “separated,” as used herein, refer to a compound, protein, cell, nucleic acid or amino acid that is removed from at least one component with which it is naturally associated.

As used herein, the term “activity” or “biological activity” refers to an activity associated with a particular protein, such as enzymatic activity. Biological activity refers to any activity that would normally be attributed to that protein by one skilled in the art.

As used herein the term “specific activity” means an enzyme unit defined as the number of moles of substrate converted to product by an enzyme preparation per unit time under specific conditions. Specific activity is generally expressed as units (U)/mg of protein.

The term “derived” encompasses the terms “originated from,” “obtained,” “obtainable from,” and “isolated from.”

The terms “host cell” or “host strain” mean a cell that contains a vector and supports the replication, and/or transcription or transcription and translation (expression) of the expression construct. In some embodiments, host cells or strains are prokaryotic, e.g., bacterial cells, including, but not limited to, Streptomyces cells.

The term “variant” refers to a region of a nucleic acid or a protein that contains one or more different, additional or less nucleotides or amino acids as compared to a reference nucleic acid or protein, for example, a naturally occurring or wild-type nucleic acid or protein. It is intended that a variant protein retains the function of the reference protein i.e. the variant protein is a functionally active variant of the reference protein. In some embodiments, the ability of the variant protein to perform said function is equal or greater than that of the reference protein.

As used herein the term “Streptomyces sp.” includes all species within the genus “Strptomyces” as known to those of skill in the art, including but not limited to S. achromogenes, S. albicans, S. albogriseolus, S. ambofaciens, S. avermitilis, S. carbophilus, S. clavuligerus, S. coelicolor, S. felleus, S. ferralitis, S. filamentosus, S. griseus, S. helvaticus S. hygroscopicus, S. lysosuperficus, S. lividans, S. noursei, S. plicatosporus, S. rubiginosus, S. scabies, S. somaliensis, S. thermoviolaceus, and S. violaceoruber.

The present teachings are based on the discovery that certain proteins can be produced in bacterial host cells using the Tat pathway. Accordingly, the present teachings provide methods for producing a protein of interest in a host cell. The present teachings also provide polynucleotides encoding a protein of interest and a Tat signal peptide sequence and fusion polypeptides encoded by the polynucleotides. In particular, the present invention relates to polynucleotides, vectors, polypeptides and methods for expressing organophosphate-degrading enzymes e.g. organophosphorus hydrolase (OPH) in host cells, such as a Streptomyces species host cells.

In one aspect, the present teachings provide a polynucleotide comprising a first nucleic acid sequence and a second nucleic acid sequence. The first nucleic acid sequence encodes a Tat signal peptide and the second nucleic acid sequence encodes a protein of interest. The first nucleic acid sequence is generally operably linked to the second nucleic acid sequence.

The Tat signal peptide encoded by the first nucleic acid can be any Tat signal peptide now known, or later discovered, that is involved in the secretion of a polypeptide via the Tat pathway. Typically, the Tat signal peptide comprises an amino acid sequence comprising a twin arginine (i.e., an “RR”) motif, wherein the RR represents two adjacent arginine amino acids. In some embodiments, the Tat signal peptide sequence comprises an RR motif within the first 5, 10, 15, 20, 25, 30, 35 or 40 amino acids from the N-terminal end of the polypeptide. In other embodiments, the Tat signal peptide sequence comprises an RR motif that is not within the first 5, 10, 15, 20, 25, 30, 35 or 40 amino acids from the N-terminal end of the polypeptide.

In some embodiments, the Tat signal peptide is a TAT2 or TAT3 signal peptide, e.g., one comprising the amino acid sequence of any of SEQ ID NO: 1-4, or a TAT3 (SEQ ID NO:5) signal peptide and the protein of interest is an esterase, for example, a phosphotriesterase. In some embodiments, the Tat signal peptide is a TAT2 or TAT3 signal peptide, or a signal peptide of protein SCO2286 (SEQ ID NO:6), SCO3790long (SEQ ID NO:7), SCO6580long (SEQ ID NO:8), SCO1590 (SEQ ID NO:9), SCO1824 (SEQ ID NO:10), SCO6580short (SEQ ID NO:11), SCO3790short (SEQ ID NO:12), SCO736 (SEQ ID NO:13), SCO2068 (SEQ ID NO:14), SCO3471 (SEQ ID NO:15), or SCO7677 (SEQ ID NO:16), and the protein of interest is OPH or a biologically active variant or fragment thereof (see, e.g., PCT Application No. GB/004816). In some embodiments, the Tat signal peptide comprises an amino acid sequence that is substantially identical, e.g., 80%, 85%, 90%, 95%, 97%, 98% 99% or 100% identical to the amino acid sequence of any of SEQ ID NO: 1 to SEQ ID NO: 16 provided in Table 1. In some embodiments, the Tat signal peptide is a TAT2 or TAT3 signal peptide. In some embodiments, the Tat signal peptide is a TAT3 signal peptide. In some embodiments, the Tat signal peptide is a TAT3 signal peptide of SEQ ID NO:5 and the protein of interest is a phosphotriesterase, for example, OPH from Flavobacterium sp. strain ATCC27551 (Mulbry et Karns J supra), Pseudomonas diminuta OPH (Munnecke supra), or Agrobacterium radiobacter (Home et al. supra) or a biologically active variant or fragment thereof.

The second nucleic acid sequence encodes a protein of interest or a fragment thereof. A protein of interest can be any protein or polypeptide, now known, or later discovered, that can be secreted by a host cell via the Tat pathway. Examples of such proteins include, but are not limited to, hydrolases, including esterases, proteases, glycosylases; isomerases, such as racemases, epimerases, tautomerases, or mutases; lyases; ligases; transferases, such as kinases, transaminases and phosphotransferases; or oxidoreductases, such as oxidases and dehydrogenases. The protein of interest can also be a therapeutically significant protein or polypeptide, such as a growth factor, cytokine, ligand, receptor or inhibitor, as well as a vaccine or an antibody. The protein of interest may be a commercially important industrial protein or peptide, such as an esterase, protease, carbohydrase such as amylase and glucoamylase, cellulase, oxidase or lipase.

The protein of interest may be a naturally occurring protein, a mutated protein or a synthetic polypeptide. The protein of interest can also be a biologically active fragment of a full-length protein. One skilled in the art can select such fragments based upon the intended activity or function of the protein of interest. In addition, the protein of interest can be a heterologous or a homologous protein. The homologous protein can comprise an amino acid sequence of the naturally occurring protein, or it can comprise a sequence comprising an insertion, deletion or interruption, as compared to the naturally occurring homologous protein.

In some embodiments, the second nucleic acid encoding the protein of interest can, but need not be, codon optimized for expression in a particular host. Codon optimization is a technique that is well-known to one of skill in the art for efficient translation of a heterologous polypeptide in a foreign host cell. Codon optimization can, for example, be performed by a commercial service, e.g., GeneArt (Toronto, Canada), that optimizes the gene encoding a particular protein of interest for expression in a host cell of choice.

In some embodiments, the protein of interest is an organophosphate-degrading enzyme. In one embodiment, the organophosphate-degrading enzyme is a phosphoric triester hydrolase (EC 3.1.8) i.e. an aryldialkylphosphatase (EC 3.1.8.1) or a diisopropyl-fluorophosphatase (EC 3.1.8.2). The aryldialkylphosphatase is also known as organophosphate hydrolase (OPH); paraoxonase; A-esterase; aryltriphosphatase; organophosphate esterase; esterase B1; esterase E4; paraoxon esterase; pirimiphos-methyloxon esterase; OPA anhydrase; organophosphorus hydrolase; phosphotriesterase; paraoxon hydrolase; OPH; or organophosphorus acid anhydrase. The diisopropyl-fluorophosphatase is also known as DFPase; tabunase; somanase; organophosphorus acid anhydrolase; organophosphate acid anhydrase; OPA anhydrase; diisopropylphosphofluoridase; dialkylfluorophosphatase; diisopropyl phosphorofluoridate hydrolase; isopropylphosphorofluoridase; and diisopropylfluorophosphonate dehalogenase. Phosphotriesterases (OPH) are members of the amidohydrolase superfamily (Seibert & Raushel, Biochemistry, 44, 6383-6391 [2005]), enzymes that catalyze the hydrolysis of a wide range of compounds with different chemical properties (phosphoesters, esters, amides etc.). The invention encompasses OPH enzymes of bacterial species including Flavobacterium sp. strain ATCC27551 (Mulbry et Karns J. Bacteriol. 171:6740-6746 [1989]), Pseudomonas diminuta OPH (Munnecke, D M. Appl. Environ. Microbiol. 32, 7-13 [1976]), and the very similar (90% sequence identity) OpdA from Agrobacterium radiobacter (Horne et al., FEMS Microbiol. Lett. 222, 1-8 [2003]). In some embodiments, the OPH has SEQ ID NO:18, or a variant thereof.

Organophosphorus hydrolase (OPH, EC 3.1.8.1) is a dimeric, bacterial enzyme that detoxifies many organophosphorus neurotoxins by hydrolyzing a variety of phosphonate bonds. Major achievements in agriculture crop production have been obtained by using pesticides for successful pest control. One of the most popular types of pesticide is the organophosphate (OP) family, which effectively eliminates pests owing to its acute neurotoxicity. The effectiveness of OP compounds as pesticides and insecticides also makes them hazardous to humans and to the environment. OPs and their family of compounds are potent neurotoxins that share structural similarities to chemical warfare agents such as sarin, soman and VX [O-ethyl-S-(2-diisopropylaminoethyl) methylphosphonothioate]. They act as cholinesterase inhibitors and in turn disrupt neurotransmission in both insects and humans. OPH is capable of hydrolyzing a variety of OP neurotoxins including common insecticides and structurally similar chemical warfare agents such as sarin and soman (Dumas et al. J boil Chem 261:19659-19665 [1989]).

While the enzyme's natural substrate and function remain unknown, it is most effective at hydrolyzing the P—O bond of the phosphotriester insecticide paraoxon, which has catalytic rates approaching the limits of diffusion (10⁸-10⁹ M⁻¹s⁻¹). The activity of native OPH varies between the different phosphonothioate substrates, exhibiting a more limited efficacy against the P—S bond of demeton-S (k_{cat}=4 s⁻¹) (Lai et al., Archives of Biochem Biophys 318:59-64 [1995]), Kolakowski et al., Biocatal. Biotransform. 15:297-312 [1997]); diSioudi et al., Chem Biol Interact 119-120:211-223 [1999]). The chemical warfare agents VX (O-ethyl S-diisopropyl aminomethyl methylphosphonothioate) and VR(O-isobutyl S—N,N-diethylaminoethyl methylphosphonothioate) belong to this class of modestly hydrolyzed compounds (P—S bonds) (Lai et al., 1995 supra; Kolakowski et al., 1997 supra; Rastogi et al., 1997 supra). In an attempt to enhance the ability of OPH to degrade these phosphonothioate nerve agents, research efforts have focused on improving the catalytic efficiency and substrate specificity of OPH by creating variants of the native OPH (Lai et al., 1996 supra; diSioudi et al., Biochemistry 38:2866-2872 [1999]). (Hill et al., Am. Chem. Soc. 125:8990-8991 [2003]). Thus, in some embodiments, the protein of interest is a variant of OPH. It is intended that the variant of OPH retains the ability to hydrolyze organophosphates.

In another embodiment, the organophosphate-degrading enzyme is a prolidase e.g. organophosphorus acid anhydrolase (OPAA) e.g. the OPAA that was identified in a strain of Alteromonas (Cheng et al., Appl. Environ. Microbiol. 59, 3138-3140 [1993]). In yet another embodiment, the organophosphate-degrading enzyme is a HDL-associated human paraoxonase (HPON) (Harel, M. et al., Nature Struct. Mol. Biol. 11:412-419 [2004]).

In some embodiments, the protein of interest is a hydrolase; an oxido reductase, e.g., an oxidase; a transferase; a lyase; an isomerase; a ligase; or a commercially or therapeutically significant protein or polypeptide. In some embodiments, the hydrolase is an esterase or a metallo-dependent hydrolase. In some embodiments, the esterase is a phosphotriesterase, for example, a phosphotriesterase classified as a EC 3.1.8 protein.

In some embodiments, the protein of interest is a metallo-dependent hydrolase associated with at least one divalent metal ion. The superfamily of metallo-dependent hydrolases (also called amidohydrolase superfamily) is a large group of proteins that show conservation in their 3-dimensional fold (TIM barrel) and in details of their active site. The vast majority of the members have a conserved metal binding site, involving four histidines and one aspartic acid residue. In the common reaction mechanism, the metal ion (or ions) deprotonate a water molecule for a nucleophilic attack on the substrate. The family includes urease alpha, adenosine deaminase, phosphotriesterase, dihydroorotases, allantoinases, hydantoinases, AMP-, adenine and cytosine deaminases, imidazolonepropionase, aryldialkylphosphatase, chlorohydrolases, formylmethanofuran dehydrogenases and others.

In some embodiments, the metallo-dependent hydrolase is OPH. OPH can functionally accommodate many different metals. The Zn²⁺ form of OPH is one of the most stable dimeric proteins ever identified, with a conformational stability of 40 kcal/mol, and the Co²⁺ form is the most active of the metal-liganded forms (Grimsley et al., Biochemistry 36:14366-14374 [1997]). Examples of divalent metal ions that can be associated with OPH include, but are not limited to, Mg²⁺, Ca²⁺, Mn²⁺, Co²⁺, Ni²⁺, Zn²⁺, and Cd²⁺. In some embodiments, the divalent metal ions include, but are not limited to, Mn²⁺, Co²⁺, Ni²⁺, Zn²⁺, and Cd²⁺. In some embodiments, the protein of interest is a metallo-dependent hydrolase that is associated with two divalent metal ions. The two divalent metal ions may be the same, or may be different. In some embodiments, the metallo-dependent hydrolase is OPH and the two divalent metal ions with which OPH is associated are selected from the group consisting of Mn²⁺, Co²⁺, Ni²⁺, Zn²⁺, Cd²⁺ and a combination thereof.

In some embodiments, the protein of interest is organophosphorus hydrolase (“OPH”), hexose oxidase (“HOX”), sorbitol oxidase (“SOX”), acetyl transferase (“ACT”), or biologically active variants or fragments thereof. In some embodiments, the protein of interest is OPH or a biologically active variant or fragment thereof.

In some embodiments, the protein of interest is OPH and the second nucleic acid sequence encoding the OPH has been codon optimized for expression of the OPH in a prokaryotic host cell. In some embodiments, the host cell is a Streptomyces sp. host cell e.g. a Streptomyces lividans host cell.

In some embodiments, the protein of interest is a hydrolase, but not a glycosylase or glycoside hydrolase. In some embodiments, the protein of interest is a hydrolase, but not an enzyme classified as a EC 3.2.1 protein. In some embodiments, the protein of interest is a hydrolase, but not a xylanaseA or a chitosanase.

In some embodiments, the polynucleotides of the invention are operably linked to a promoter. The polynucleotides can be linked to any suitable promoter now known, or later discovered, in the art. The promoter can be native or heterologous to the prokaryotic host in which the protein of interest is expressed. Examples of promoter include, but are not limited to, the Aspergillus niger A4 promoter (herein SEQ ID NO:23), the Aspergillus niger A4 long promoter (A4-long promoter), the Aspergillus niger A4-5′ promoter (U.S. Patent Publication 2006/0105425), the glucose isomerase (GI) promoter of Actinoplanes missouriensis and the derivative GI (GIT) promoter (U.S. Pat. No. 6,562,612 and EPA 351029); the glucose isomerase (GI) promoter from Streptomyces lividans (SEQ ID NO: 1), the short wild-type GI promoter, the 1.5 GI promoter, the 1.20 GI promoter, or any of the variant GI promoters as disclosed in WO 03/089621, the aph promoter of the Streptomyces fradiae aminoglycoside 3′-phosphotransferase gene, ssi promoter, and Streptomyces lividans xylanase xlnA promoter. In some embodiments, the polynucleotide of the invention that comprises a first nucleic acid encoding a TAT signal peptide operably linked to a second nucleic acid encoding a phosphoric trimester hydrolase classified as EC 3.1.8 is operably linked to the A4 promoter of SEQ ID NO:23.

(SEQ ID NO: 23)
TGCCGGCTTCTCTGTGGGCTTCGGCCCCTCTGGCCCAATGGCTAGCGG
AGCAAACTCCCGATCGAACTTCATGTTCGAGTTCTTGTTCACGTAGAA
GCCGGAGATGTGAGAGGTGATCTGGAACTGCTCACCCTCGTTGGTGGT
GACCTGGAGGTAAAGCAAGTGACCCTTCTGGCGGAGGTGGTAAGGAAC
GGGGTTCCACGGGGAGAGAGAGATGGCCTTGACGGTCTTGGGAAGGGG
AGCTTCGGCGCGGGGGAGGATGGTCTTGAGAGAGGGGGAGCTAGTAAT
GTCGTACTTGGACAGGGAGTGCTCCTTCTCCGACGCATCAGCCACCTC
AGCGGAGATGGCATCGTGCAGAGACAGAC

TABLE 1
Sequences of Exemplary Signal Peptides and Proteins of
Interest and the Nucleic Acid Sequences Encoding Them

| Name | Sequence | Sequence ID |
|---|---|---|
| TAT2 | MGTEVSRRKLMKGAAVSGGALALPALGAPPATAAP | SEQ ID NO: 1 |
| | AAGPEDLPGPAAA | |
| TAT2 | MGTEVSRRKLMKGAAVSGGALALPALGAPPATAAP | SEQ ID NO: 2 |
| alternate 1 | AAGPEDLPGPAAAAA | |
| TAT2 | MTEVSRRKLMKGAAVSGGALALPALGAPPATAAPA | SEQ ID NO: 3 |
| alternate 2 | AGPEDLPGPAAA | |
| TAT2 | MTEVSRRKLMKGAAVSGGALALPALGAPPATAAPA | SEQ ID NO: 4 |
| alternate 3 | AGPEDLPGPAAAAA | |
| TAT3: | MHEPHLDRRLFLKGTAVTGAALALGATAAPTASAA | SEQ ID NO: 5 |
| SCO2286 | PMTPANHQAPTSAPSPAPSQSSHAPELRAAARSLG | SEQ ID NO: 6 |
| signal peptide | RRRFLTVTGAAAALAFAVNLPAAGTASAAE | |
| SCO3790 long | MRKLLPLIGTPSGSHPGGRSAMTCRFRCGDACFHE | SEQ ID NO: 7 |
| signal peptide | VPNTSSNEYVGDVIAGALSRRSMMRAAAVVTVAAA | |
| | GAGAVGVAGAPSAQAAP | |
| SCO6580 long | MTPFTDSSRTDAGTDPSADGPGESLRRALGVNRRR | SEQ ID NO: 8 |
| signal peptide | FLSTCTAVAAGAVAAPVFGASPALAHDR | |
| SCO1590 | MGGVSRRAFTVAALSAFTLVPEASAATP | SEQ ID NO: 9 |
| signal peptide | | |
| SCO1824 | MTAPLSRHRRALAIPAGLAVAASLAFLPGTPAAATP | SEQ ID NO: 10 |
| signal peptide | AAEAAP | |
| SCO6580 | MNRRRFLSTCTAVAAGAVAAPVFGASPALAHDR | SEQ ID NO: 11 |
| short signal | | |
| peptide | | |
| SCO3790 | MPNTSSNEYVGDVIAGALSRRSMMRAAAVVTVAAA | SEQ ID NO: 12 |
| short signal | GAGAVGVAGAPSAQAAP | |
| peptide | | |
| SCO736 signal | MGDIRRRGAVALGVTALVAPLTLALTAAPAQAASC | SEQ ID NO: 13 |
| peptide | | |
| SCO2068 | MTSRHRASENSRTPSRRTVVKAAAAGAVLAAPLAAA | SEQ ID NO: 14 |
| signal peptide | LPAGAADAAP | |
| SCO3471 | MVNRRDLIKWSAVALGAGAGLAGPAPAAHAADL | SEQ ID NO: 15 |
| signal peptide | | |
| SCO7677 | MSRQIDRRSFLRRGAAGAAALAVGPGLLAACSTDEP | SEQ ID NO: 16 |
| signal peptide | GSAGNPG | |
| TAT1 signal | MQTRRVVLKSAAAAGTLLGGLAGCASVAGS | SEQ ID NO: 17 |
| peptide | | |
| OPH | IGTGDRINTVRGPITISEAGFTLTHEHICGSSAGFLR | SEQ ID NO: 18 |
| | AWPEFFGSRKALAEKAVRGLRRARAAGVRTIVDVSTF | |
| | DIGRDVSLLAEVSRAADVHIVAATGLWFDPPLSMRLR | |
| | SVEELTQFFLREIQYGIEDTGIRAGIIKVATTGKATP | |
| | FQELVLKAAARASLATGVPVTTHTAASQRDGEQQAAI | |
| | FESEGLSPSRVCIGHSDDTDDLSYLTALAARGYLIGL | |
| | DHIPHSAIGLEDNASASALLGIRSWQTRALLIKALID | |
| | AGYMKQILVSNDWLFGFSSYVTNIMDVMDRVNPDGMA | |
| | FIPLRVIPFLREKGVPQETLAGITVTNPARFLSPTLR | |
| | AS | |
| TAT1 DNA | ATGCAAACGAGAAGGGTTGTGCTCAAGTCTGCGG | SEQ ID NO: 19 |
| | CCGCCGCAGGAACTCTGCTCGGCGGCCTGGCTGG | |
| | GTGCGCGAGCGTGGCTGGATCG | |
| TAT2 DNA | ATGGGCACCGAGGTCTCCCGCCGCAAGCTGATGA | SEQ ID NO: 20 |
| | AGGGCGCCGCCGTCAGCGGCGGCGCCCTGGCCCT | |
| | GCCGGCCCTGGGCGCCCCGCCGGCCACCGCCGCC | |
| | CCGGCCGCCGGCCCGGAGGACCTGCCGGGCCCGG | |
| | CCGCCGCG | |
| TAT3 DNA | ATGCACGAGCCGCACCTCGACCGCCGTCTGTTCC | SEQ ID NO: 21 |
| | TGAAGGGCACGGCCGTCACCGGCGCCGCCCTCG | |
| | CACTGGGCGCCACCGCCGCGCCCACCGCCTCCG | |
| | CCGCCCCC | |
| OPH Gene that | ATCGGCACCGGCGACCGCATCAACACGGTCCGCG | SEQ ID NO: 22 |
| has been | GCCCGATCACCATCTCCGAGGCCGGCTTCACCCT | |
| Codon | GACCCACGAGCACATCTGCGGCTCCTCCGCCGGC | |
| Optimized for | CTTCCTGCGCGCCTGGCCGGAGTTCTTCGGCTCC | |
| Expression in | CGCAAGGCCCTGGCCGAGAAGGCCGTCCGCGGCC | |
| Streptomyces | TGCGCCGCGCCCGGGCCGCCGGCGTCCGCACCAT | |
| | CGTGGACGTGTCCACCTTCGACATCGGCCGCGAC | |
| | GTGTCCCTGCTGGCCGAGGTCTCGCGCGCCGCCG | |
| | ACGTCCACATCGTCGCCGCCACCGGCCTGTGGTT | |
| | CGACCCGCCGCTGTCCATGCGCCTGCGCTCCGTC | |
| | GAGGAGCTGACCCAGTTCTTCCTCCGCGAGATCC | |
| | AGTACGGCATCGAGGACACCGGCATCCGCGCCGG | |
| | CATCATCAAGGTCGCCACCACCGGCAAGGCCACC | |
| | CCGTTCCAGGAGCTGGTCCTGAAGGCCGCCGCCC | |
| | GCGCCTCCCTGGCCACCGGCGTCCCGGTCACACC | |
| | CACACCGCCGCCTCCCAGCGCGACGGCGAGCAGC | |
| | AGGCCGCCATCTTCGAGTCCGAGGGCCTGTCCCC | |
| | GTCCCGCGTCTGCATCGGCCACTCCGACGACACC | |
| | GACGACCTGTCCTACCTGACCGCCCTGGCCGCCC | |
| | GCGGCTACCTGATCGGCCTGGACCACATCCCGCA | |
| | CTCCGCCATCGGCCTGGAGGACAACGCCTCCGCC | |
| | TCCGCCCTGCTGGGCATCCGCTCGTGGCAGACCC | |
| | GCGCCCTGCTGATCAAGGCCCTGATCGACCAGGG | |
| | CTACATGAAGCAGATCCTGGTCTCCAACGACTGG | |
| | CTGTTCGGCTTCTCCTCCTACGTCACCAACATCA | |
| | TGGACGTCATGGACCGCGTCAACCCGGACGGCAT | |
| | GGCCTTCATCCCGCTGCGCGTGATCCCGTTCCTG | |
| | CGCGAGAAGGGCGTCCCGCAGGAGACCCTGGCCG | |
| | GCATCACCGTGACCAACCCGGCCCGCTTCCTGTC | |
| | CCCGACCCTGCGCGCCTCCTGA | |

In another aspect, the present teachings provide a fusion polypeptide comprising a Tat signal peptide and a protein of interest of the invention. Any one of the TAT signal peptides recited herein are fused to the protein of interest. In some embodiments, the invention provides an isolated fusion polypeptide that comprises a TAT2 e.g. SEQ ID NOS:1-4 or a TAT3 e.g. SEQ ID NO:5 signal peptide operably linked to the organophosphorus hydrolase of SEQ ID NO:18 or variant thereof. In other embodiment, the TAT signal peptide is chosen from SEQ ID NO:1 or 5.

The fusion polypeptide optionally includes a linker peptide located between the TAT signal peptide and the protein of interest e.g. OPH. The linker peptide can be of any suitable length, including, but not limited to, 1, 2, 3, 4, 5, 6, 7, 8, 9, 10, 15, 20, 25, 30, 35, 40, 45 or 50 amino acids. In some embodiments, the linker peptide comprises between 1 and 10 amino acids, or 1 and 5 amino acids. In some embodiments, the linker peptide comprises 1 or 2 amino acids. The linker peptide can comprise any naturally-occurring, or modified amino acid. In some embodiments, the linker peptide comprises an alanine. In some embodiments, the linker is a cleavable linker and is easily cleaved to release the mature protein from the pre-protein that additionally comprises the signal peptide.

In some embodiments, the present teachings provide an expression vector containing the polynucleotides of the invention. The vector can be any vector now known, or later discovered, that would be suitable for expressing the protein of interest in the host cell. The vector can be an integrating vector or a replicating vector. In general, the vector contains an origin of replication functional in at least one organism, convenient restriction endonuclease sites, and a selectable marker for the host cell. Examples of vectors that can be used include, but are not limited to, pKB105, pKB229, pKB231, pKB233, and pKB234. In some embodiment, the expression vector comprises an isolated polynucleotide comprising a first nucleic acid sequence encoding a TAT signal peptide operably linked to a second nucleic acid sequence encoding a phosphoric triester hydrolase classified as a EC 3.1.8 e.g. organophosphorus hydrolase protein. In some embodiments, the expression vector comprises a first nucleic acid sequence encoding a TAT signal peptide of any one of SEQ ID NOS:1-5 and a second nucleic acid sequence encoding a phosphoric triester hydrolase. In some embodiment, the isolated polynucleotide comprising a first nucleic acid sequence encoding a TAT signal peptide that is operably linked to a second nucleic acid sequence encoding a phosphoric triester hydrolase is further operably linked to the A4 promoter of SEQ ID NO:23. In other embodiments, the nucleic acid sequence encoding the phosphoric triester hydrolase is codon optimized for expression in a Streptomyces sp. host cell. In some embodiments, the Streptomyces sp. host cell is a Streptomyces lividans host cell.

In some embodiments, the present teachings provide a host cell containing the polynucleotides of the invention. The host cell can be any cell, for example a higher eukaryotic, lower eukaryotic or a prokaryotic cell, known in the art that is capable of expressing the protein of interest and secreting it via the Tat pathway. In some embodiments, the host cell is a prokaryotic cell, for example, a bacterial cell. The bacterial host cell may be from gram positive or gram negative bacteria. In some embodiments, the host cell is a from gram positive bacteria. A number of types of gram positive cells that may act as suitable host cells for production of the protein of interest include, for example, Streptomyces, Bacillus and Lactococcus cells.

In some embodiments, the host cell is a Streptomyces cell. As used herein, the genus Streptomyces includes all members known to those skilled in the art, including but not limited to S. achromogenes, S. albicans, S. albogriseolus, S. ambofaciens, S. avermitilis, S. carbophilus, S. clavuligerus, S. coelicolor, S. felleus, S. ferralitis, S. filamentosus, S. griseus, S. helvaticus S. hygroscopicus, S. lysosuperficus, S. lividans, S. noursei, S. plicatosporus, S. rubiginosus, S. scabies, S. somaliensis, S. thermoviolaceus, and S. violaceoruber. In some embodiments, the host cell is a Bacillus cell, including, but not limited to, a B. clausii, B. subtilis, B. licheniformis, and B. lentus cell. In some embodiments, the host cell is a S. albogriseolus, S. carbophilus, S. coelicolor, S. lividans, S. rubiginosus, S. helvaticus or a B. subtilis cell.

In yet another aspect, the present teachings provide a method of producing a protein of interest in a host cell. The method comprises expressing a polynucleotide of the invention in the host cell. As discussed above, the host cell can be any suitable cell, for example, a prokaryotic cell or a bacterial cell, including, but not limited to, a Streptomyces or Bacillus cell. In some embodiments, the protein of interest is produced in a folded form. In some embodiments, the protein of interest is produced in its active form. In some embodiments, the host cell of the invention e.g. a Streptomyces sp. host cell, comprises an expression vector comprising an isolated polynucleotide comprising a first nucleic acid sequence encoding a TAT signal peptide operably linked to a second nucleic acid sequence encoding a phosphoric triester hydrolase classified as a EC 3.1.8 e.g. organophosphorus hydrolase protein. In some embodiments, the expression vector comprises a first nucleic acid sequence encoding a TAT signal peptide of any one of SEQ ID NOS:1-5 and a second nucleic acid sequence encoding a phosphoric triester hydrolase. In another embodiment, the expression vector comprises an isolated polynucleotide comprising a first nucleic acid sequence encoding a TAT signal peptide that is operably linked to a second nucleic acid sequence encoding a phosphoric triester hydrolase is further operably linked to the A4 promoter of SEQ ID NO:23. In other embodiments, the nucleic acid sequence encoding the phosphoric triester hydrolase is codon optimized for expression in a Streptomyces sp. host cell. In some embodiments, the host cell of the invention is a Streptomyces sp. host cell e.g. a Streptomyces lividans host cell.

In some embodiments, the host cells and transformed cells of the present invention are cultured in conventional nutrient media. The suitable specific culture conditions, such as temperature, pH and the like are known to those skilled in the art. In addition, some preferred culture conditions may be found in the scientific literature such as Hopwood (2000) Practical Streptomyces Genetics, John Innes Foundation, Norwich UK; Hardwood et al., (1990) Molecular Biological Methods for Bacillus, John Wiley and from the American Type Culture Collection (ATCC).

In some embodiments, host cells transformed with polynucleotide sequences encoding the TAT-fusion proteins e.g. TAT-OPH fusion proteins are cultured under conditions suitable for the expression and recovery of the encoded protein from cell culture. The protein produced by a recombinant host cell is secreted into the culture media. In some embodiments, other recombinant constructions join the heterologous polynucleotide sequences encoding the TAT-OPH proteins to a nucleotide sequence encoding a polypeptide domain which facilitates purification of the soluble proteins (Kroll D J et al (1993) DNA Cell Biol 12:441-53).

Such purification facilitating domains include, but are not limited to, metal chelating peptides such as histidine-tryptophan modules that allow purification on immobilized metals (Porath J (1992) Protein Expr Purif 3:263-281), protein A domains that allow purification on immobilized immunoglobulin, and the domain utilized in the FLAGS extension/affinity purification system (Immunex Corp, Seattle Wash.). The inclusion of a cleavable linker sequence such as Factor XA or enterokinase (Invitrogen, San Diego Calif.) between the purification domain and the heterologous protein also find use to facilitate purification.

In some preferred embodiments, the transformed host cells of the present invention are cultured in a suitable nutrient medium under conditions permitting the expression of the present OPH, after which the resulting OPH is recovered from the culture. The medium used to culture the cells comprises any conventional medium suitable for growing the host cells, such as minimal or complex media containing appropriate supplements. Suitable media are available from commercial suppliers or may be prepared according to published recipes (e.g., in catalogues of the American Type Culture Collection). In some embodiments, the OPH produced by the cells is recovered from the culture medium by conventional procedures, including, but not limited to separating the host cells from the medium by centrifugation or filtration, precipitating the proteinaceous components of the supernatant or filtrate by means of a salt (e.g., ammonium sulfate), chromatographic purification (e.g., ion exchange, gel filtration, affinity, etc.). Thus, any method suitable for recovering the OPH of the present invention finds use in the present invention. Indeed, it is not intended that the present invention be limited to any particular purification method.

In some embodiments, the present teachings provide a method of producing a protein of interest in a host cell. The method comprises expressing a polynucleotide of the invention in the host cell, culturing the host cell in a medium and recovering the protein of interest from the medium.

The methods of the invention may be practiced and the expressed protein can be isolated on a small scale or on a larger, e.g., industrial scale. In some embodiments, the present teachings provide a protein of interest produced by the methods of the invention. In some embodiments, the protein of interest is in a folded form or in its active form.

Aspects of the present teachings may be further understood in light of the following examples, which should not be construed as limiting the scope of the present teachings. It will be apparent to those skilled in the art that many modifications, both to materials and methods, may be practiced without departing from the present teachings.

### EXPERIMENTAL

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); kg (kilograms); μg (micrograms); L (liters); ml (milliliters); μl (microliters); cm (centimeters); mm (millimeters); μm (micrometers); nm (nanometers); ° C. (degrees Centigrade); h (hours); min (minutes); sec (seconds); msec (milliseconds); TY, trypton/yeast extract; Ap, ampicillin; DTT, dithiotreitol; Em, erythromycin; HPDM, high phosphate defined medium; MM, minimal medium; OD, optical density; PAGE, polyacrylamide gel electrophoresis; PCR, polymerase chain reaction.

### Example 1

### Construction of the Expression Plasmids for OPH Production in Streptomyces Host Cells

The OPH protein expressed in the Streptomyces lividans g3s3 host cells comprised the following amino acid sequence:

(SEQ ID NO: 18)
IGTGDRINTVRGPITISEAGFTLTHEHICGSSAGFLRAWPEFFGSRK
ALAEKAVRGLRRARAAGVRTIVDVSTFDIGRDVSLLAEVSRAADVHI
VAATGLWFDPPLSMRLRSVEELTQFFLREIQYGIEDTGIRAGIIKVA
TTGKATPFQELVLKAAARASLATGVPVTTHTAASQRDGEQQAAIFES
EGLSPSRVCIGHSDDTDDLSYLTALAARGYLIGLDHIPHSAIGLEDN
ASASALLGIRSWQTRALLIKALIDQGYMKQILVSNDWLFGFSSYVTN
IMDVMDRVNPDGMAFIPLRVIPFLREKGVPQETLAGITVTNPARFLS
PTLRAS

And was encoded by the corresponding OPH gene (SEQ ID NO:22) that was synthesized by the GeneArt Inc. (Toronto, Canada) company with codon optimization for Streptomyces genes.

(SEQ ID NO: 22)
ATCGGCACCGGCGACCGCATCAACACGGTCCGCGGCCCGATCACCAT
CTCCGAGGCCGGCTTCACCCTGACCCACGAGCACATCTGCGGCTCCT
CCGCCGGCTTCCTGCGCGCCTGGCCGGAGTTCTTCGGCTCCCGCAAG
GCCCTGGCCGAGAAGGCCGTCCGCGGCCTGCGCCGCGCCCGGGCCGC
CGGCGTCCGCACCATCGTGGACGTGTCCACCTTCGACATCGGCCGCG
ACGTGTCCCTGCTGGCCGAGGTCTCGCGCGCCGCCGACGTCCACATC
GTCGCCGCCACCGGCCTGTGGTTCGACCCGCCGCTGTCCATGCGCCT
GCGCTCCGTCGAGGAGCTGACCCAGTTCTTCCTCCGCGAGATCCAGT
ACGGCATCGAGGACACCGGCATCCGCGCCGGCATCATCAAGGTCGCC
ACCACCGGCAAGGCCACCCCGTTCCAGGAGCTGGTCCTGAAGGCCGC
CGCCCGCGCCTCCCTGGCCACCGGCGTCCCGGTCACCACCCACACCG
CCGCCTCCCAGCGCGACGGCGAGCAGCAGGCCGCCATCTTCGAGTCC
GAGGGCCTGTCCCCGTCCCGCGTCTGCATCGGCCACTCCGACGACAC
CGACGACCTGTCCTACCTGACCGCCCTGGCCGCCCGCGGCTACCTGA
TCGGCCTGGACCACATCCCGCACTCCGCCATCGGCCTGGAGGACAAC
GCCTCCGCCTCCGCCCTGCTGGGCATCCGCTCGTGGCAGACCCGCGC
CCTGCTGATCAAGGCCCTGATCGACCAGGGCTACATGAAGCAGATCC
TGGTCTCCAACGACTGGCTGTTCGGCTTCTCCTCCTACGTCACCAAC
ATCATGGACGTCATGGACCGCGTCAACCCGGACGGCATGGCCTTCAT
CCCGCTGCGCGTGATCCCGTTCCTGCGCGAGAAGGGCGTCCCGCAGG
AGACCCTGGCCGGCATCACCGTGACCAACCCGGCCCGCTTCCTGTCC
CCGACCCTGCGCGCCTCCTGA

The following polynucleotides encoding signal sequences were used to express OPH in Streptomyces lividans host cells:

1. Truncated celA signal sequence;
2. ASP signal sequence;
3. TAT1: OPH signal sequence codon optimized for expression in Streptomyces

(SEQ ID NO: 19)
ATGCAAACGAGAAGGGTTGTGCTCAAGTCTGCGGCCGCCGCAGGAA
CTCTGCTCGGCGGCCTGGCTGGGTGCGCGAGCGTGGCTGGATCG;

4. TAT2: modified putative signal peptide of SCO6272

(SEQ ID NO: 20)
ATGGGCACCGAGGTCTCCCGCCGCAAGCTGATGAAGGGCGCCGCC
GTCAGCGGCGGCGCCCTGGCCCTGCCGGCCCTGGGCGCCCCGCCG
GCCACCGCCGCCCCGGCCGCCGGCCCGGAGGACCTGCCGGGCCCG
GCCGCCGCG;

and
5. TAT3: putative signal peptide of SCO624

(SEQ ID NO: 21)
ATGCACGAGCCGCACCTCGACCGCCGTCTGTTCCTGAAGGGCACGG
CCGTCACCGGCGCCGCCCTCGCACTGGGCGCCACCGCCGCGCCCAC
CGCCTCCGCCGCCCCC

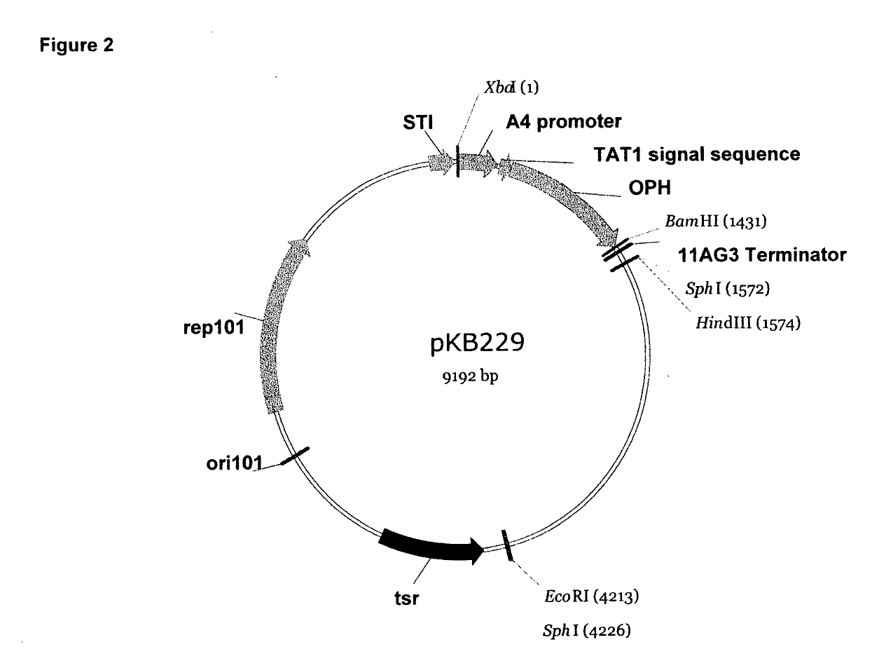

Plasmid pKB229 was constructed from plasmid pKB105 by replacing the segment encoding the celA signal peptide and the cellulase catalytic core with a fusion polynucleotide sequence encoding OPH signal peptide (TAT1; SEQ ID NO:19) and OPH protein (SEQ ID NO:18). See FIG. 2.

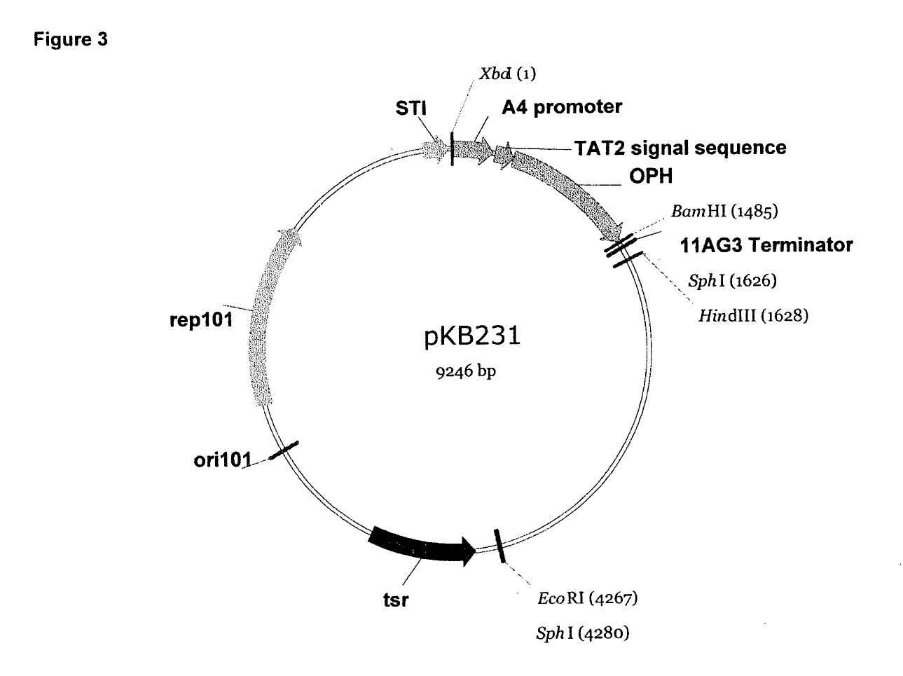

Plasmid pKB231 was constructed from plasmid pKB105 by replacing the segment encoding the celA signal peptide and the cellulase catalytic core with a fusion polynucleotide sequence (SEQ ID NO:20) encoding the TAT2 signal peptide (SEQ ID NO:1) and OPH protein (SEQ ID NO:18). See FIG. 3.

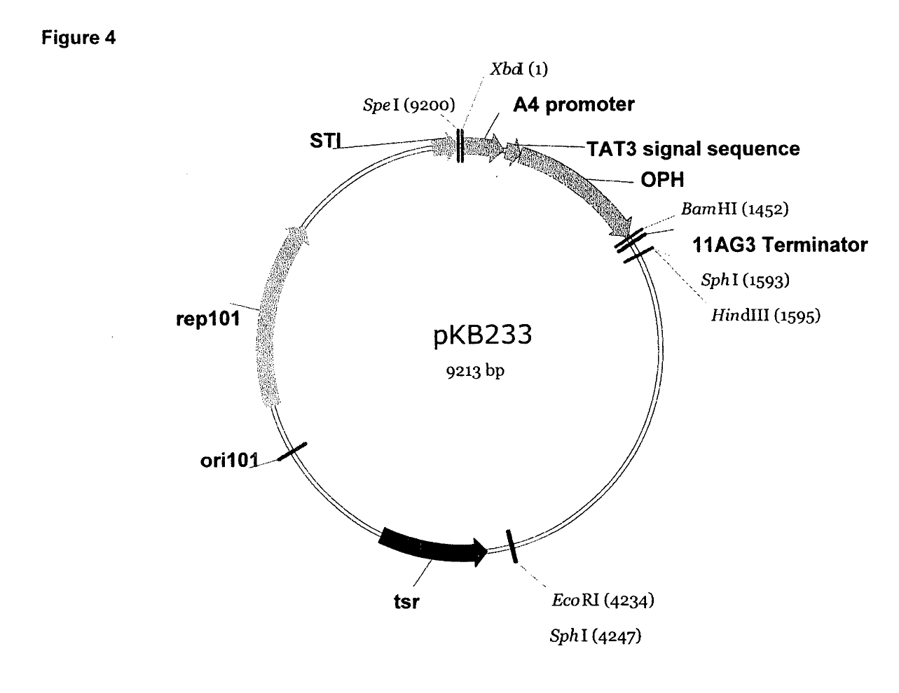

Plasmid pKB233 was constructed from plasmid pKB105 by replacing the segment encoding the celA signal peptide and the cellulase catalytic core with a fusion polynucleotide sequence (SEQ ID NO:21) encoding the TAT3 signal peptide (SEQ ID NO:5) and OPH protein (SEQ ID NO:18). See FIG. 4.

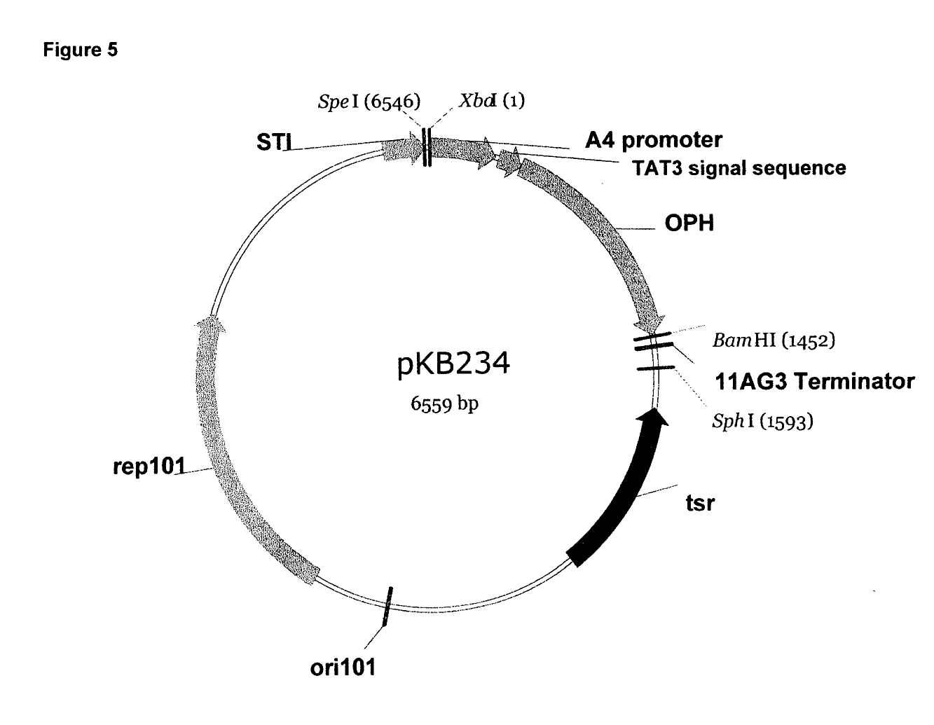

For the production of OPH in fermentor, the expression vector, pKB234, was derived from pKB233 by removing E. coli DNA sequences in pKB233. To remove the E. coli sequences, pKB233 was digested with SphI, EcoRI and HindIII overnight at 37° C. The digested DNA was purified using a Qiagen kit and then re-ligated for transformation of Streptomyces host cells. FIG. 5 shows the pKB234 vector.

### Example 2

### Expression and Activity of OPH Produced by Streptomyces Host Cells

The following example describes the effect of various TAT signal peptides on the expression and activity of OPH .

### Transformation and Expression:

The expression vectors, pKB229, pKB231 and pKB233, as described above, were used in this example.

In these experiments, the host Streptomyces lividans cells were transformed with the vectors described above. The transformation techniques were the protoplast method described in Hopwood, et at., GENETIC MANIPULATION OF STREPTOMYCES, A LABORATORY MANUAL. The John Innes Foundation, Norwich, United Kingdom (1985).

Streptomyces lividans cells were transformed with one of the expression vectors described above. Transformed cells were plated on R5 plates (R5 plate for 1 liter: 206 g sucrose, 0.5 g K.sub.2SO.sub.4, 20.24 g MgCL.sub.2, 20 g glucose, 0.2 g Difco casamino acids, 10 g Difco yeast extracts and 11.46 g TES, 4 g L-Asp, 4 ml of trace elements and 44 g Difco agar in 800 ml H₂O. 20 ml 5% K.sub.2HPO.sub.4 and 8 ml 5M CaCL.sub.2.2H.sub.20 and 14 ml 1N NaOH were added to a final 1 liter after autoclaving. Transformants were grown in 20 ml of TSG in 250 ml shake flasks for 2 days in the presence of 50 μg/ml thiostrepton at 30° C. Cells were then transferred to a production medium (50 ml) free of antibiotics and growth was continued for another three days. Samples were taken for electrophoretic and enzyme activity assays.

TSG media contained 16 g Difco tryptone, 4 g Difco soytone, 20 g caseine (hydrolysate) sigm and 5g K₂HPO₄ brought to 1 liter. After autoclaving 50% filtered sterilized glucose was added to a final concentration of 1.5%.

Production Media: 2.4 g Citric Acid.H₂O; 8.3 g Biospringer Yeast Extract; 2.4 g (NH₄)₂SO₄; 72.4 g MgSO₄.7H₂O; 0.1 g CaCl₂.2H₂O; 0.3 ml Mazu DF204 (antifoam); 5 ml Streptomyces modified trace elements (1 liter stock solution contains: 250 g Citric acid.H₂O; 3.25 g FeSO₄.7H₂O; 5 g ZnSO₄.7H₂O; 5 g MnSO₄.H₂O; 0.25 g H₃BO₃); 10 g glucose, adjust volume to 1 liter. Adjust pH to 6.9 with NaOH. In some experiments, the production media was supplemented with either Zn²⁺ or Co²⁺.

### Recovery

One ml of sample of the cell culture was taken from each shake flask and centrifuged at 14,000 rpm to sediment the cells. The OPH enzyme secreted into the culture medium was analyzed by SDS-PAGE electrophoresis and the activity of the enzyme was tested as described below.

### OPH Activity Assay and SDS-PAGE Analysis

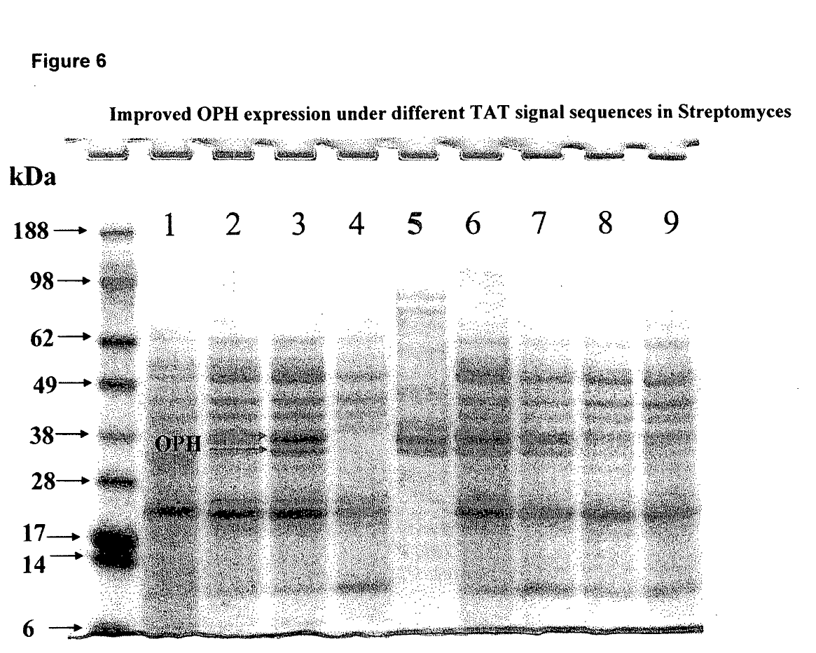

(A) OPH production with the above vectors in S. lividans cells (Lanes 1-4 and 6-9) was analyzed by SDS-PAGE analysis as shown in FIG. 6. The double arrow indicates that OPH migrates as a doublet under non-reducing conditions. Lane 1: direct OPH expression under A4 promoter without any signal peptide; Lane 2: OPH expression with TAT1 signal sequence; Lane 3: OPH expression with TAT3 signal sequence; Lane 4: Expression backbone with only A4 promoter (served as negative control); Lane 5: OPH expression in E. coli as inclusion body (served as positive control); Lane 6: same as lane 3; Lane 7: same as lane 3 except for the addition of cobalt (at a final concentration of 0.5 mM) into the production medium; Lane 8: OPH expression with TAT2 signal sequence, cobalt was added into production at the final concentration of 0.5 mM; Lane 9: OPH expression with TAT1 signal sequence with cobalt at a final concentration of 0.5 mM.

The results show that the production of OPH by bacterial host cells is greater when OPH is expressed as a TAT2-OPH or a TAT3-OPH fusion protein as compared to the production of TAT1-OPH. Expression of the TAT3-OPH fusion polynucleotide lead to the greatest production of OPH by Streptomyces host cells.

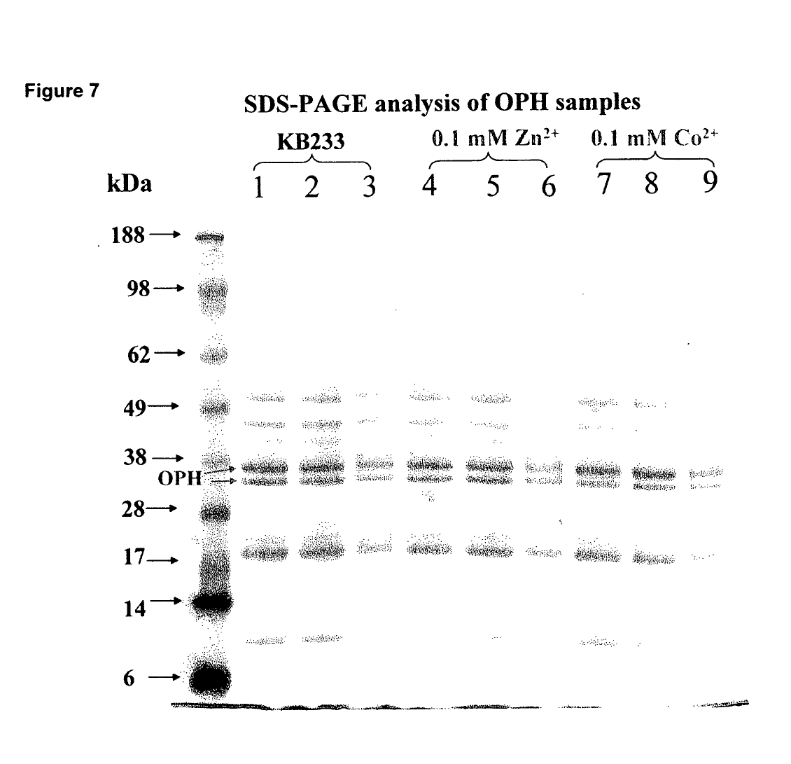

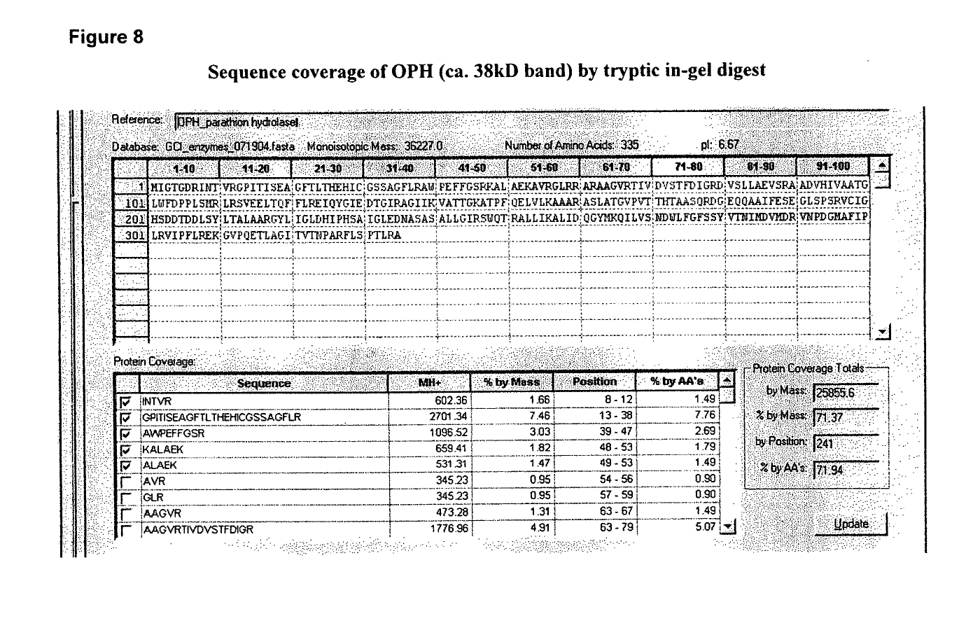

(B) The effect of addition of Zn²⁺ and Co²⁺ into the production medium fermentation on OPH production was examined. FIG. 7 shows the effect of addition Zn²⁺ and Co²⁺ on OPH production and storage stability of OPH when expressed with a TAT3 signal peptide. Lanes 1, 2 and 3 show the production level of OPH expressed as TAT3-OPH following storage at 4° C., −20° C. and −20° C. with 20% glycerol, respectively. Lanes 4, 5, and 6 show the production level of OPH expressed as TAT3-OPH in the presence of 0.1 mM Zn²⁺ following storage at 4° C., −20° C. and −20° C. with 20% glycerol, respectively. Lanes 7, 8, and 9 show the production level of OPH expressed as TAT3-OPH in the presence of 0.1 mM Co²⁺ following storage at 4° C., −20° C. and −20° C. with 20% glycerol, respectively. The sequence of the expected OPH band was confirmed by MS peptide mapping, and is shown in FIG. 8.

These data indicate that the production level of OPH is similar whether in the presence or absence of metal ions, and that OPH is best stored either at 4° C., or at −20° C.

(C) Samples corresponding to samples 1-9 shown in FIG. 7 were tested for activity (Caldwell et al. Biochemistry 30:7438-7444 [1991]; Rastogi et al., Biochem Biophys Res Commun 241:294-296 [1997]). As the paraoxon and VX substrates used in the OPH activity assays are highly toxic, the supernatants of the different shake flask samples were sent out to the U.S. Army (US Army-ECBC, AMSRD-ECB-RT-BP, E-3150 Kingscreek St. N. APG, MD 21010) for specific OPH activity assay.

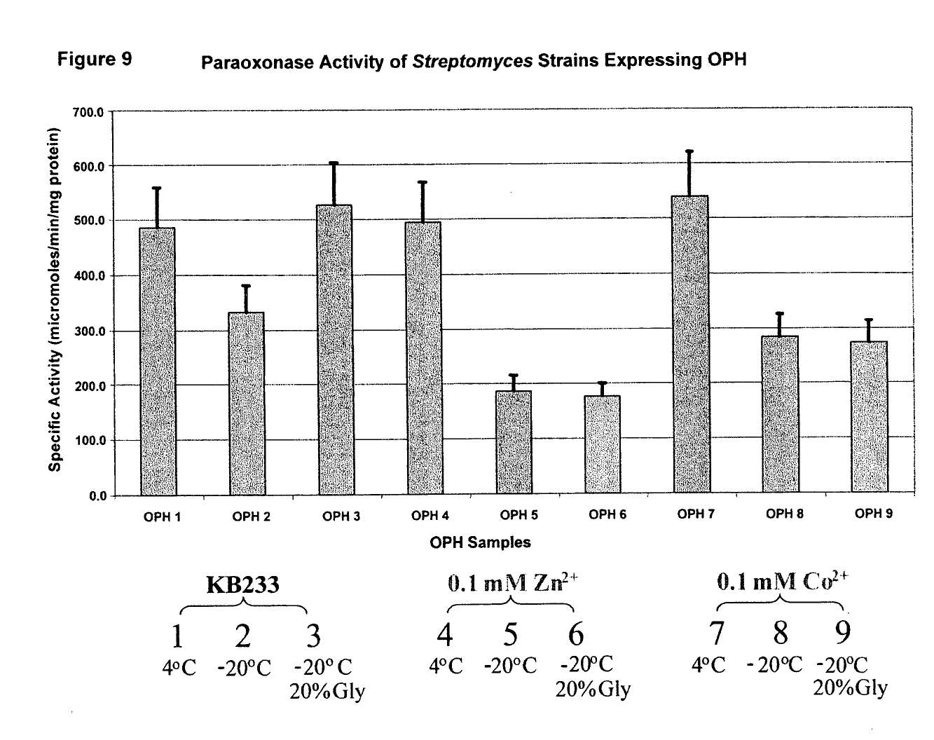

The OPH specific activity assay results are shown in FIG. 9. Bars shown as OPH1-9 correspond to the samples in lanes 1-9 of FIG. 7. These data show that the OPH expressed as a fusion protein with the TAT3 signal peptide and produced by the Streptomyces host cells retains organophosphate degrading activity.

We claim:

1. An isolated polynucleotide comprising a first nucleic acid sequence encoding a TAT signal peptide operably linked to a second nucleic acid sequence encoding a phosphoric triester hydrolase classified as a EC 3.1.8 protein.

2. The isolated polynucleotide of claim 1, wherein said TAT signal peptide comprises an amino acid sequence chosen from SEQ ID NOS:1-5.

3. The isolated polynucleotide of claim 1, wherein said hydrolase is an organophosphorus hydrolase (OPH).

4. The isolated polynucleotide of claim 1, wherein said hydrolase is the organophosphorus hydrolase (OPH) of SEQ ID NO:18, or a variant thereof.

5. The isolated polynucleotide of claim 1, wherein said second nucleic acid encoding said hydrolase is optimized for expressing said hydrolase in a Streptomyces sp. host cell.

6. The isolated polynucleotide of claim 1, wherein said isolated polynucleotide is operably linked to the A4 promoter of SEQ ID NO:23.

7. An expression vector comprising the isolated polynucleotide of claim 1.

8. An isolated host cell comprising the expression vector of claim 7.

9. The isolated host cell of claim 8, wherein said host cell is a Streptomyces host cell.

10. An isolated fusion polypeptide comprising a TAT2 or a TAT3 signal peptide linked to the organophosphorus hydrolase of SEQ ID NO:18 or variant thereof.

11. The isolated fusion polypeptide of claim 10, wherein said TAT signal peptide is chosen from SEQ ID NO:1 or 5.

12. A method for producing an organophosphate degrading enzyme comprising:
(a) expressing the polynucleotide of claim 1 in a host cell; and
(b) producing said enzyme.

13. The method of claim 12, wherein said enzyme produced by said host cell is recovered.

14. The method of claim 12, wherein said host cell is a Streptomyces host cell.

15. The method of claim 12, wherein said enzyme is organophosphorus hydrolase.
